(19) United States
(12) Patent Application Publication
(10) Pub. No.: US 2008/0306260 A1
(43) Pub. Date: Dec. 11, 2008

(54) Method for Producing Aryl Amines, Aryl Ethers and Aryl Thioethers

(76) Inventors: Andreas Meudt, Hofheim (DE); Sven Nerdinger, Kiefersfelden (DE); Bernd Wilhelm Lehnemann, Frankfurt am Main (DE); Joerg Jung, Floersheim (DE); Till Vogel, Mannheim (DE); Victor Snieckus, Kingston (CA)

Correspondence Address: PROPAT, L.L.C., 425-C SOUTH SHARON AMITY ROAD, CHARLOTTE, NC, 28211-2841, US

(21) Appl. No.: 11/917,928

(22) PCT Filed: Jun. 14, 2006
(86) PCT No.: PCT/EP2006/005719

§ 371 Date: Aug. 8, 2008

(30) Foreign Application Priority Data
Jun. 27, 2005 (DE) 10 2005 030 400.1

(51) Int. Cl. C07D 295/00; C07C 211/43; C07C 229/00; C07C 39/00; C07F 9/02; C07F 15/00

(52) U.S. Cl. 544/106; 564/428; 560/34; 568/716; 568/13; 556/13

(57) ABSTRACT

The invention relates to a method for producing aryl or heteroaryl amines, ethers or thioethers (III) by cross-coupling primary or secondary amities, alcohols or thioalcohols with substituted aryl or heteroaryl compounds (I) in the presence of a Brønsted base and a catalyst or a pre-catalyst containing a) a transition metal, a complex, a salt, or a compound of a transition metal from the group V, Mn, Fe, Co, Ni, Rh, Pd, Ir, Pt, and b) at least one sulfonated phosphane ligand in a solvent or a solvent mixture corresponding to Scheme 1 wherein Hal represents fluorine, chlorine, bromine, iodine, alkoxy, trifluoromethane sulfonate, nonafluorotrimethyl-methane sulfonate, methane sulfonate, 4-nitrobenzene sulfonate, benzene sulfonate, 2-naphthalene sulfonate, 3-nitrobenzene sulfonate, 4-nitrobenzene sulfonate, 4-chlorobenzene sulfonate, 2,4,6-triisopropylbenzene sulfonate or any other sulfonate, and X represents O, S or NR″. The invention also relates to novel phosphane ligands.

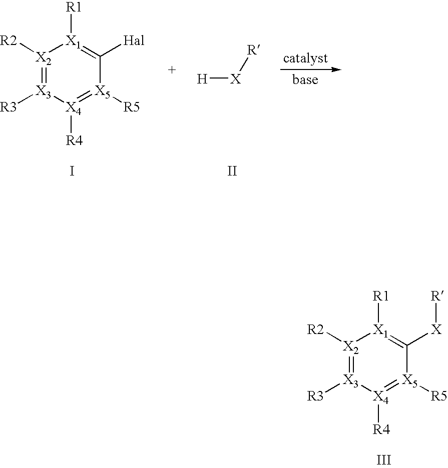

Mixed aryl- or heteroaryl-substituted alkyl-/arylamines and aryl- or heteroaryl-substituted alkyl/aryl ethers, in particular having functional groups in the alkyl chain, are important and extremely versatile intermediates in organic synthesis. Their significance in modern organic synthesis is restricted only by limitations in the availability of this compound class. The standard process for preparing mixed aryl- or heteroaryl-substituted alkyl-/arylamines and aryl- or heteroaryl-substituted alkyl/aryl ethers is the Ullmann reaction, but the reaction requires very high temperatures to proceed to completion. However, these generally severe reaction conditions are rarely tolerated by functional groups and reactive heteroatoms, and can be applied to electron-deficient aromatics only with very great difficulty, if at all, and can additionally be controlled only with difficulty. More modern processes for preparing these amines and ethers use Pd- or Ni-catalyzed couplings of amines or alcohols in the presence of various ligands. However, the currently known processes all have process technology or economic disadvantages which considerably restrict the scope of application. These include high costs of the catalysts/ligands, high required loadings/catalyst concentrations and difficult removability of the catalyst from the end product. One reason for the latter is that the ligands used to date are all substantially nonpolar and, as a result, preferably remain in the organic phase in aqueous workups.

It would be very desirable to have a process which can convert substituted alkyl- or arylamines, alcohols or phenols and haloaromatics or haloheteroaromatics to the corresponding mixed aryl- or heteroaryl-substituted alkyl-/arylamines and aryl- or heteroaryl-substituted alkyl/aryl ethers, simultaneously achieves very high yields, works with very small amounts of catalyst and is additionally notable for simple removal of the ligand and of the transition metal used from the product. As already mentioned, the synthesis methods published for this purpose to date do not satisfactorily solve this problem, as will be demonstrated further with reference to a few examples:
Use of expensive ligands (e.g. P^{t}Bu₃, Hartwig et al., U.S. Pat. No. 6,100,398) and complicated isolation of the product by chromatography.
Use of ligands which are difficult to synthesize (ferrocene-based ligands, Hartwig et al., WO 02/11883), complicated isolation of the product by chromatography.
Complicated or difficult, often multistage ligand syntheses (Buchwald et al., WO 00/02887), complicated isolation of the product by chromatography.

Further methods for C—X bond formation (X═O, N, S) from aryl halides or sulfonates using various catalysts feature the following disadvantages (Wolfe, J. P.; Buchwald, S. L. J. Org. Chem. 2000, 65, 1444; Wolfe, J. P.; Buchwald, S. L. J. Org. Chem. 2000, 65, 1158; Huang, J.; Grassa, G.; Nolan, S. P. Org. Lett. 1999, 1, 1307; Hartwig, J. F.; Kawatsura, M.; Hauck, S. I.; Shaughnessy, K. H.; Alcazar-Roman, L. M. J. Org. Chem. 1999, 64, 5575; Stauffer, S. I.; Hauck, S. I.; Lee, S.; Stambuli, J.; Hartwig, J. F. Org. Lett. 2000, 2, 1423):
The reaction temperatures are in many cases very high, which often causes side reactions and low selectivities.
For C—N bond formations, the selectivities for the formation of the desired anilines, in contrast to the undesired amines or diarylamines, are often too low for economic application.
The removal of the catalyst from the product is often difficult, since the amines formed bind the transition metals quite effectively, but, on the other hand, very low specification limits should be complied with especially for fine pharmaceutical chemicals (e.g. <10 or <5 ppm). In addition, the catalyst systems used customarily are highly active in various other reactions, such that undesired side reactions can also be catalyzed in subsequent stages.

The present process solves all of these problems and relates to a process for preparing aryl- and heteroaryl amines, aryl- or heteroaryl-substituted alkyl/aryl ethers or aryl- or heteroaryl-substituted alkyl/aryl thioethers (III) by cross-coupling primary or secondary alkyl- or arylamines, alcohols or phenols, or thioalcohols or thioethers (II) with substituted aryl or heteroaryl compounds (I), in the presence of a Brønsted base and of a catalyst or precatalyst comprising
a.) a transition metal, a complex, salt or a compound of this transition metal from the group of {V, Mn, Fe, Co, Ni, Rh, Pd, Ir, Pt}, and
b.) at least one sulfonated phosphine ligand in a solvent or solvent mixture, according to scheme 1.

The process according to the invention features the following advantages:
In the case of very low catalyst loadings, high yields and very high selectivities are achieved.
It offers a simple and economically viable route to sulfonated ligands by sulfonating commercially available or easily obtainable ligands (example: the 2-hydroxy-2′ dialkylphosphinobiaryls which are obtainable in a simple and very economically viable manner according to U.S. Pat. No. 5,789,623 can be converted to the corresponding sulfonated ligands by simple treatment with sulfuric acid. As a result of the obtainability/availability of the corresponding oxaphosphorin chlorides (e.g. 10-chloro-10H-9-oxa-10-phosphaphenanthrene), the reaction is overall a very simple two-stage reaction which proceeds with good yields and is notable for very high flexibility, since a wide variety of different radicals can be introduced in a very simple manner on the phosphorus.)
The catalyst activities achieved by the process according to the invention are very high, since the ligand is present in the reaction mixture as an anion and as a result has particular electronic effects (on this subject, see especially example 13).
Fine tuning of the electronic properties of the inventive ligands is possible through the possibility of different counterions (metal cations, substituted ammonium salts, etc.). Especially in the case of ligands which can be deprotonated twice, for example in the case of sulfonated 2-hydroxy-2′-dialkylphosphinobiphenyls, it is possible here to tailor them very previously to the particular requirements of a particular reaction.
Simple removal of the ligand and metal from the product by aqueous extraction, since, as a result of the very high acidity/polarity of the sulfonated ligands, they preferably reside in the aqueous phase.
The reaction can also be performed in protic solvents, for example substituted alcohols, with an often positive influence on the selectivity/reactivity.
As a result of the additionally finely adjustable parameters mentioned, the process according to the invention widens the scope of application of the C—X coupling technologies known to date to an exceptional degree.
Exceptional activity of the sulfonated ligand/catalyst systems (cf. example 13), as a result often rapid reactions and short reaction times

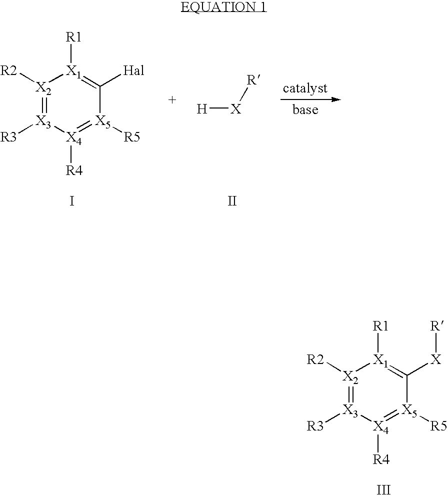

In equation 1, Hal is fluorine, chlorine, bromine, iodine, alkoxy, or sulfonate leaving groups, for example trifluoromethanesulfonate (triflate), nonafluorotrimethylmethanesulfonate (nonaflate), methanesulfonate, benzenesulfonate, para-toluenesulfonate,

### X is O, S or NR″,

X₁₋₅ are each independently carbon, or XᵢRᵢ are each nitrogen, or in each case two adjacent XᵢRᵢ bonded via a formal double bond together are C (furans), S (thiophenes), NH or NRᵢ (pyrroles).

Preferred compounds of the formula (I) which can be converted by the process according to the invention are, for example, benzenes, pyridines, pyrimidines, pyrazines, pyridazines, furans, thiophenes, pyrroles, pyrroles or naphthalenes with any N-substitution, quinolines, indoles, benzofurans, etc.

The R₁₋₅ radicals are substituents from the group of hydrogen, methyl, primary, secondary or tertiary, cyclic or acyclic alkyl radicals having from 2 to 20 carbon atoms, in which one or more hydrogen atoms are optionally replaced by fluorine or chlorine or bromine, e.g. CF₃, substituted cyclic or acyclic alkyl groups hydroxyl, alkoxy, amino, alkylamino, dialkylamino, arylamino, diarylamino, alkylarylamino, pentafluorosulfanyl, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻, alkyl or aryloxycarbonyl, hydroxyalkyl, alkoxyalkyl, fluorine or chlorine, nitro, cyano, aryl- or alkylsulfone, aryl- or alkylsulfonyl, or it is possible in each case for two adjacent R₁₋₅ radicals together to correspond to an aromatic, heteroaromatic or aliphatic fused-on ring.

When X═O or S, R′ may be identical or different radicals from the group of {hydrogen, methyl, linear, branched C₁-C₂₀ alkyl or cyclic alkyl, substituted or unsubstituted aryl or heteroaryl}.

When X═NR″, R′ and R″ may each independently be identical or different radicals from the group of {hydrogen, methyl, linear, branched C₁-C₂₀ alkyl or cyclic alkyl, substituted or unsubstituted aryl or heteroaryl) or together form a ring.

Typical examples of the compound II are thus methyl, ethyl, 1-methylethyl, propyl, 1-methylpropyl, 2 methylpropyl, 1,1-dimethylethyl, butyl and pentylamine, cyclopropyl, cyclobutyl, cyclopentyl, cyclohexylamine, phenyl, benzylamine, morpholine, and also tert-butanol, isopropanol, neopentyl alcohol or n-alkanols, phenol or thiophenol.

The catalyst used in accordance with the invention is a transition metal, preferably on a support, for example palladium on carbon, or a salt, a complex or an organometallic compound of this metal selected from the group of {V, Mn, Fe, Co, Ni, Rh, Pd, Ir, Pt}, preferably palladium or nickel, with a sulfonated ligand. The catalyst may be added in finished form or be formed in situ, for example from a precatalyst by reduction or hydrolysis or from a metal salt and added ligand by complex formation. The catalyst is used in combination with one or more, but at least one, sulfonated phosphorus-containing ligand. The metal may be used in any oxidation state. According to the invention, it is used in relation to the reactant I in amounts of from 0.0001 mol % to 100 mol %, preferably between 0.01 and 10 mol %, more preferably between 0.01 and 1 mol %.

According to the invention, sulfonated phosphine ligands which feature the presence of at least one sulfonic acid group or a salt of a sulfonic acid group in the molecule are used.

Preference is given to using ligands of the structure (IV) shown below

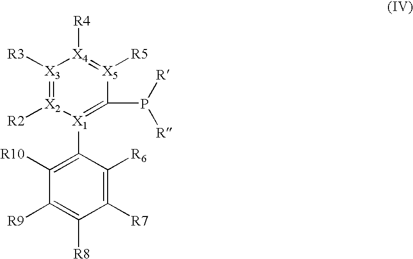

in conjunction with transition metals, preferably palladium or nickel, as the catalyst.

In this structure,

X₁ is carbon or nitrogen, X₂₋₅ are each independently carbon, or XᵢRᵢ is nitrogen, or in each case two adjacent XᵢRᵢ bonded via a formal double bond, where i=2, 3, 4, 5, together are O (furans), S (thiophenes), NH or NRᵢ (pyrroles);
the R₂₋₁₀ radicals, where at least one radical is a sulfonic acid or sulfonate group, are each substituents from the group of {hydrogen, methyl, primary, secondary or tertiary, cyclic or acyclic alkyl radicals having from 2 to 20 carbon atoms, in which one or more hydrogen atoms are optionally replaced by fluorine or chlorine or bromine, e.g. CF₃, substituted cyclic or acyclic alkyl groups, hydroxyl, alkoxy, amino, alkylamino, dialkylamino, arylamino, diarylamino, alkylarylamino, pentafluorosulfanyl, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻; alkyl- or aryloxycarbonyl, hydroxyalkyl, alkoxyalkyl, nitro, cyano, aryl- or alkylsulfone, aryl- or alkylsulfonyl}, or in each case two adjacent R₁₅ radicals together are an aromatic, heteroaromatic or aliphatic fused-on ring;

R′ and R″ are each independently identical or different radicals from the group of {hydrogen, methyl, linear, branched or cyclic C₁-C₂₀-alkyl, optionally substituted, phenyl, optionally substituted} or together form a ring and are a bridging structural element from the group of {optionally substituted alkylene, branched alkylene, cyclic alkylene}, or are each independently one or two polycyclic radicals, for example norbornyl or adamantyl.

Particular preference is given here to those derivatives which, as well as at least one sulfonic acid group, also contain a further deprotonatable function in the molecule, for example a free OH group in the sulfonated ring.

In a further preferred embodiment, complexes of a sulfonated secondary phosphine are used in conjunction with a palladacycle as a catalyst of the structure

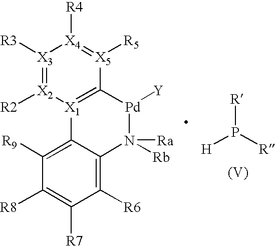

where the symbols X₁₋₅, R₂₋₉, R′ and R″ are each as defined above and Y is a radical from the group of {halide, pseudohalide, alkylcarboxylate, trifluoroacetate, nitrate, nitrite}, and R^{a} and R^{b} are each independently identical or different substituents from the group of {hydrogen, methyl, primary, secondary or tertiary, optionally substituted C₁-C₂₀-alkyl or aryl} or together form a ring and stem from the group of {optionally substituted alkylene, oxaalkylene, thiaalkylene, azaalkylene},

and at least one sulfonic acid group or a sulfonate salt is present in the secondary phosphine.

In a further preferred embodiment, complexes of a tertiary phosphine of the structure

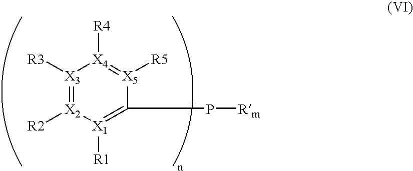

are used, where the symbols X₁₋₅, R₁₋₅ and R′ are each as defined above, where n may be 1, 2 or 3 and m=3-n, and the n aryl or heteroaryl radicals may each independently be of identical or different nature, as may the m radicals independently be of identical or different nature, at least one sulfonated aromatic ring being present. Mixtures of different ligands of this class may be used.

The present invention further relates to novel sulfonated ligands of the formulae (IV), (VII) and (VIII) of the structures shown below, which are outstandingly suitable for the preparation of catalysts for use in organochemical synthesis

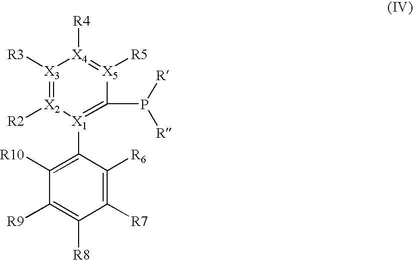

where X₁ is carbon or nitrogen, X₂₋₅ are each independently carbon, or XᵢRᵢ is nitrogen, or in each case two adjacent XᵢRᵢ bonded via a formal double bond, where i=2, 3, 4, 5, together are O (furans), S (thiophenes), NH or NRᵢ (pyrroles),
and the R₂₋₁₀ radicals, where at least one radical is a sulfonic acid or sulfonate group, are each substituents from the group of {hydrogen, methyl, primary, secondary or tertiary, cyclic or acyclic alkyl radicals having from 2 to 20 carbon atoms, in which one or more hydrogen atoms are optionally replaced by fluorine or chlorine or bromine, substituted cyclic or acyclic alkyl groups, hydroxyl, alkoxy, amino, alkylamino, dialkylamino, arylamino, diarylamino, alkylarylamino, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻, alkyl- or aryloxy carbonyl, hydroxyalkyl, alkoxyalkyl, nitro, cyano, aryl- or alkylsulfone, aryl- or alkylsulfonyl}, or in each case two adjacent R₁₋₅ radicals together are an aromatic, heteroaromatic or aliphatic fused-on ring, R′ and R′″ are each independently identical or different radicals from the group of {hydrogen, methyl, linear, branched or cyclic alkyl, phenyl} or together form a ring and are a bridging structural element from the group of (alkylene, branched alkylene, cyclic alkylene), or are each independently one or two polycyclic radicals, for example norbornyl or adamantyl.

In a further embodiment, the invention relates to sulfonated ligands of the structure

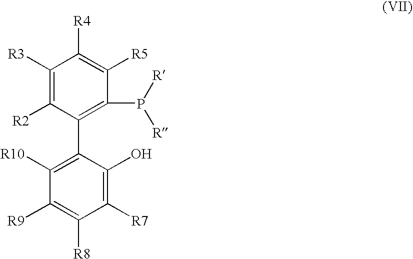

in which at least one Rᵢ radical represents a sulfonic acid or sulfonate group and the R₂₋₅ and R₇₋₁₀ radicals are each substituents from the group of {hydrogen, methyl, primary, secondary or tertiary, cyclic or acyclic alkyl radicals having from 2 to 20 carbon atoms, in which one or more hydrogen atoms are optionally replaced by fluorine or chlorine or bromine, substituted cyclic or acyclic alkyl groups, hydroxyl, alkoxy, amino, alkylamino, dialkylamino, arylamino, diarylamino, alkylarylamino, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻, alkyl or aryloxycarbonyl, hydroxyalkyl, alkoxyalkyl, nitro, cyano, aryl- or alkylsulfone, aryl- or alkylsulfonyl), or in each case two adjacent R₁₋₅ radicals together are an aromatic, heteroaromatic or aliphatic fused-on ring,
R′ and R″ are each independently identical or different radicals from the group of {hydrogen, methyl, linear, branched or cyclic alkyl, phenyl} or together form a ring and are a bridging structural element from the group of {alkylene, branched alkylene, cyclic alkylene}, or are each independently one or two polycyclic radicals, for example norbornyl or adamantyl.

The present invention likewise relates to novel sulfonated ligands of the structure

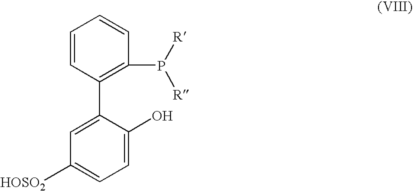

in which R′ and R″ are each independently identical or different radicals from the group of {hydrogen, methyl, linear, branched or cyclic alkyl, phenyl} or together form a ring and are a bridging structural element from the group of {alkylene, branched alkylene, cyclic alkylene} or are each independently one or two polycyclic radicals, for example norbornyl or adamantyl.

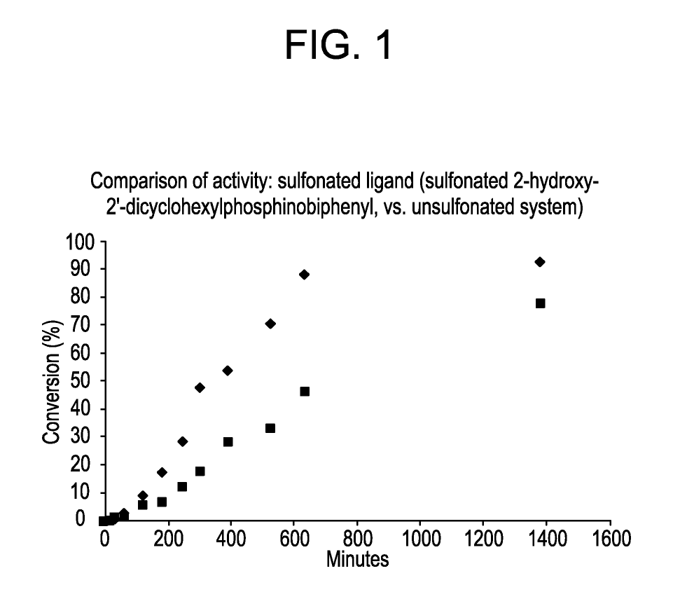

Suitable catalysts or precatalysts for the process according to the invention are, for example, complexes of palladium or nickel with sulfonated biaryl-phosphines, some of which are obtainable in a very simple and economically viable manner (for example TV and V, for preparation cf. EP 0 795 559), or, as representatives of the third type described, the commercially available sulfonated triphenylphosphines TPPTS, TPPDS and TPPMS VI a-c (FIG. 1).

The addition of Brønsted bases to the reaction mixture is necessary to achieve acceptable reaction rates. Very suitable bases are, for example, hydroxides, alkoxides and fluorides of the alkali metals and alkaline earth metals, carbonates, hydrogencarbonates and phosphates of the alkali metals, and mixtures thereof. Particularly suitable bases are those from the group of {potassium tert-butoxide, sodium tert-butoxide, cesium tert-butoxide, lithium tert-butoxide and the corresponding isopropoxides}. It is customary to use at least the amount of base which corresponds to the amount of the amine, phenol or alcohol II to be coupled; usually from 1.0 to 6 equivalents, preferably from 1.2 to 3 equivalents, of base are used, based on the compound (II).

The reaction is performed in a suitable solvent or a monophasic or polyphasic solvent mixture which has a sufficient dissolution capacity for all reactants involved, heterogeneous performance also being possible (for example use of almost insoluble bases). Preference is given to performing the reaction in polar, aprotic or protic solvents. Very suitable solvents are open-chain and cyclic ethers and diethers, oligo- and polyethers, and substituted mono- or polyalcohols and optionally substituted aromatics. Particular preference is given to using one solvent or a mixture of a plurality of solvents from the group of {diglyme, substituted glymes, 1,4-dioxane, isopropanol, tert-butanol, 2,2-dimethyl-1-propanol, toluene, xylene}.

The reaction can be performed at temperatures between room temperature and the boiling point of the solvent used at the pressure used. In order to achieve a more rapid reaction, performance at elevated temperatures in the range from 0 to 240° C. is preferred. Particular preference is given to the temperature range from 20 to 200° C., especially from 50 to 150° C.

The concentration of the reactants can be varied within wide ranges. Appropriately, the reaction is performed at a maximum concentration, for which the solubilities of the reactants and reagents in the particular reaction medium have to be taken into account. Preference is given to performing the reaction in the range between 0.05 and 5 mol/l based on the reactant present in deficiency (depending on the relative costs of the reactants).

Amine, alcohol, phenol, thioalcohol or thiophenol of the formula (II) and aromatic or heteroaromatic reactants (I) may be used in molar ratios of from 10:1 to 1:10; particular preference is given to ratios of from 3:1 to 1:3 and particular preference to ratios of from 1.2:1 to 1:1.2.

In one of the preferred embodiments, all materials are initially charged and the mixture is heated to reaction temperature with stirring. In a further preferred embodiment which is suitable particularly for application on a large scale, the compound (II) and optionally further reactants, for example base and catalyst or precatalyst, are metered into the reaction mixture during the reaction. Alternatively, can also be performed under metering control by slow addition of the base.

The workup is effected typically with a mixture of aromatic hydrocarbons/water with removal of the aqueous phase which takes up the inorganic constituents and also ligand and transition metal, the product remaining in the organic phase unless acidic functional groups which are present lead to a different phase behavior. If appropriate, ionic liquids may be used to remove the more polar constituents. The product is preferably isolated from the organic phase by precipitation or distillation, for example by concentration or by addition of precipitants. Usually, an additional purification or subsequent removal of transition metal or ligand, for example by recrystallization or chromatography, is unnecessary. The isolated yields are usually in the range from 60 to 100%, preferably in the range from >75 to 100%, especially from >80 to 100%. According to the invention, the selectivities are very high; it is usually possible to find conditions under which, apart from very small amounts of dehalogenation product, no further by-products are detectable.

The process according to the invention opens up, in the workup and removal of catalyst/ligand in particular, a very economic method of preparing mixed aryl- and heteroarylamines and aryl- or heteroaryl-substituted alkyl/aryl ethers or thioethers proceeding from the corresponding primary or secondary alkyl- or arylamines, alcohols or phenols, thioalcohols or thiophenols or derivatives thereof and the corresponding aryl or heteroaryl halides or aryl or heteroaryl sulfonates, and affords the products generally in very high purities without complicated purification procedures.

The process according to the invention will be illustrated by the examples which follow, without restricting the invention thereto:

### EXAMPLE 1

### Preparation of the ligand 2′-hydroxy-2-dicyclohexylphosphinobiphenyl-4′-sulfonic acid (HBPNS)

1.099 g (3.0 mmol) of 2-hydroxy-2′-diphenylphosphino biphenyl was precooled in an ice bath under a protective gas atmosphere. Subsequently, 2.0 ml of concentrated sulfuric acid were metered in slowly from a syringe. After warming to room temperature, the suspension formed was stirred for a further approx. 2 hours until all solid had dissolved. A homogeneous, viscous and slightly brownish suspension was obtained. The reaction mixture was cooled again in an ice bath and then quenched with ice. Concentrated sodium hydroxide solution was used to completely dissolve the precipitate formed. After dilution with 75 ml of water and acidification with 1 N sulfuric acids the precipitate was filtered off and washed with water until the washing water effluent exhibited neutral pH. The white filtercake was washed once more with methanol and dried under reduced pressure. 1.093 g (2.45 mmol 82%) of 2-hydroxy-2′-diphenylphosphinobiphenyl-5-sulfonic acid were obtained as white crystals.

### Spectroscopic Data

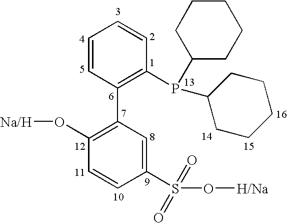

Melting point (free acid):

285-295° C. (decomposition).

¹H NMR (D₂O/NaOH) (sodium salt):

δ/ppm=0.903-1.201 (m, 10H, 5xCH₂); 1.439-1.726 (m, 10H, 5xCH₂); 1.782-1.852 (m, 2H, 2xCH); 6.526 (d, J=8.16 Hz, 11-CH); 7.210-7.300 (m, 3H, 3,4,8-CH); 7.344 (d, J=5.95 Hz, 1H, 2-CH); 7.418 (d, J=7.67 Hz, 1H, 10-CH); 7.547

(d, J=5.25 Hz, 1H, 5-CH).

¹³C NMR (D₂O/NaOH) (sodium salt):

δ/ppm=26.0, 26.1, 26.7, 26.8, 26.9, 27.0, 27.1, 27.2, 29.2, 29.3, 29.5, 29.8, 30.1 and 30.3 (14,15,16-CH₂); 33.2 (d, J=8 Hz) and 34.2 (d, J=9 Hz, 13, 13′-CH); 119.0 (II-C); 125.0 (9-C); 126.3 (4-CH); 128.6 (2-CH); 129.6 (3-CH); 131.5 (d, J=5 Hz, 8-CH); 132.0 (d, j 7 Hz, 1-C); 132.7 (5-CH); 134.3 (d, J=9 Hz, 6-C); 148.6 (d, J=27 Hz, 7-C); 168.3 (12-C).

³¹P NMR (D₂O/NaOH) (sodium salt):

δ=−10.6 ppm.

HRMS (C₂₄H₃₁O₄PS) (free acid)

calculated: 485.1318 (M+K)

found: 485.1314 (M+K)

IR (KBr) (free acid):

ν/cm⁻¹=3445, 3062, 2946, 2857, 1604, 1415, 1233, 1168, 1112, 1029, 1012, 832, 675, 593.

UV/VIS (NaOEH, 1 N, c=1*10⁻⁴ M):

λ (max)=302 nm (ε: 4032)

ε (max) 18670 (λ: 224 nm)

### EXAMPLE 2

### Coupling of 2-bromo-4-fluorotoluene with 2,3-dimethylaniline to give 5-fluoro-2,2′,3′-trimethyl-diphenylamine

189 mg (1 mmol) of 2-bromo-4-fluorotoluene, 121 mg (1 mmol) of 2,3-dimethylaniline, 192 mg (2 mmol) of sodium tert-butoxide, 4.4 mg of palladium(II) acetate (2 mol %) and 26.8 mg of the HBPNS ligand (6 mol %) were heated to 120° C. in 6 ml of degassed anhydrous diglyme for 15 h. After cooling, the reaction mixture was added to 10 ml of water and the mixture was extracted with 10 ml of toluene. To remove diglyme residues, the toluene phase was washed with 5 ml of water and concentrated on a rotary evaporator. After drying under reduced pressure, 207 mg (0.90 mmol, 90%) of the product were obtained.

### EXAMPLE 3

### Coupling of 1-bromonaphthalene with 2,3-dimethylaniline to give (2,3-dimethylphenyl)naphthalen-1-ylamine

The experiment was performed as described above, except that 20 mg of 1-bromonaphthalene (1 mmol) was used in place of 2-bromo-4-fluorotoluene, and 14 mg of tris(dibenzylideneacetone)dipalladium(0) (1.5 mol, 3 mol % of Pd) was used in place of palladium(II) acetate. The yield was 210 mg (0.85 mmol, 85%).

### EXAMPLE 4

### Coupling of 1-bromonaphthalene with 4-aminobenzonitrile to give (4-cyanophenyl)naphthalen-1-ylamine

The experiment was performed as described above, except that 118 mg of 4-aminobenzonitrile (1 mmol) were used in place of the dimethylaniline. The amount of catalyst was reduced to 2.2 mg of palladium(II) acetate (1 mol %) and the amount of ligand was reduced to 8.0 mg (1.8 mol %). A yield of 181 mg (0.74 mmol, 74%) was obtained.

### EXAMPLE 5

### Coupling of 3-bromo-4-fluorotoluene with 4-aminobenzonitrile to give 4′-cyano-2-fluoro-5-methyldiphenylamine

189 mg (1 mmol) of 3-bromo-4-fluorotoluene, 118 mg of 4-aminobenzonitrile (1 mmol), 192 mg (2 mmol) of sodium tert-butoxide, 2.2 mg of palladium(II) acetate (1 mol %) and 4.5 mg of the HBPNS ligand (1 mol %) were heated to reflux in 6 ml of degassed tert-butanol for 30 h. After cooling, the reaction mixture was added to 10 ml of water and the mixture was extracted with 10 ml of toluene. To remove tert-butanol residues, the toluene phase was washed with 5 ml of water and concentrated on a rotary evaporator. After drying under reduced pressure, 178 mg (0.79 mmol, 79%) of the product were obtained.

### EXAMPLE 6

### Coupling of 1-chloronaphthalene with 4-aminobenzonitrile to give (4-cyanophenyl)naphthalen-1-ylamine

118 mg of 4-aminobenzonitrile (1 mmol), 163 mg of 1-chloronaphthalene (1 μmol), 192 mg (2 mmol) of sodium tert-butoxide, 2.2 mg of palladium(II) acetate (1 mol %) and 4.5 mg of the HBPNS ligand (1 mol %) were heated to 120° C. in 6 ml of degassed diglyme for 15 h. After cooling, the reaction mixture was added to 10 ml of water and the mixture was extracted with 10 ml of toluene. To remove solvent residues, the toluene phase was washed with 5 ml of water and concentrated on a rotary evaporator. After drying under reduced pressure, 215 mg (0.89 mmol, 89 t) of the product were obtained.

### EXAMPLE 7

### Coupling of 1-chloronaphthalene with 4-aminobenzonitrile to give (4-cyanophenyl)naphthalen-1-ylamine

The coupling was performed as described above, except that the solvent used was tert-butanol in place of diglyme. After reaction at reflux temperature for 30 hours, the yield was 201 mg (0.82 mmol, 82%).

### EXAMPLE 8

### Coupling of 1-bromonaphthalene with morpholine to give 4-naphthalen-1-ylmorpholine

87 mg of anhydrous morpholine (1 mmol), 207 mg of 1-bromonaphthalene (1 mmol), 192 mg (2 mmol) of sodium tert-butoxide, 2.2 mg of palladium(II) acetate (1 mol %) and 4.5 mg of the HBPNS ligand (1 mol %) were heated to 120° C. in 6 ml of degassed diglyme for 24 h. After cooling, the reaction mixture was added to 10 ml of water and the mixture was extracted with 10 ml of toluene. To remove solvent residues, the toluene phase was washed with 5 ml of water and concentrated on a rotary evaporator. After drying under reduced pressure, 156 mg (0.73 mmol, 73%) of the product were obtained.

### EXAMPLE 9

### Coupling of 1-chloronaphthalene with morpholine to give 4-naphthalen-1-ylmorpholine

The reaction was performed as described in the preceding example; the bromonaphthalene was replaced by 163 mg of 1-chloronaphthalene (1 mmol). 147 mg (0.69 mmol, 69%) of the product were obtained.

### EXAMPLE 10

### Coupling of 1-bromonaphthalene with tert-butyl carbazate to give tert-butyl N′-naphthalen-1-ylhydrazinecarboxylate

The reaction was performed like example 8; instead of morpholine, 132 mg of tert-butyl carbazate (1 mmol) were used. The yield of product was 201 mg (0.8 mmol, 78%).

### EXAMPLE 11

### Coupling of 4-bromotoluene with 4-methoxyphenol to give 1-methoxy-4 (4-methylphenoxy)benzene

124 mg of 4-methoxyphenol (1 mmol), 171 mg of 4-bromotoluene (1 mmol), 192 mg (2=mol) of sodium tert-butoxide, 2.2 mg of palladium(II) acetate (1 mol %) and 4.5 mg of the HBPNS ligand (1 mol %) were heated to 120° C. in 6 ml of degassed diglyme for 24 h. After cooling; the reaction mixture was added to 10 ml of water and the mixture was extracted with 10 ml of toluene. To remove solvent residues, the toluene phase was washed with 5 ml of water and concentrated on a rotary evaporator. After drying under reduced pressure, a yield of 141 mg (0.66 mmol, 66%) was obtained.

### EXAMPLE 12

### Coupling of 2-bromotoluene with 4-methoxyphenol to give 1-methoxy-4-(2-methylphenoxy)benzene

124 mg of 4-methoxyphenol (1 mmol), 171 my of 2 bromotoluene (1 mmol), 192 mg (2 mmol) of sodium tert-butoxide, 2.2 my of palladium(II) acetate (1 mol %) and 4.5 mg of the HBPNS ligand (1 mol) were heated to 120° C. in 6 ml of degassed diglyme for 24 h. After cooling, the reaction mixture was added to 10 ml of water and the mixture was extracted with 10 ml of toluene. To remove solvent residues, the toluene phase was washed with 5 ml of water and concentrated on a rotary evaporator. After drying under reduced pressure, a yield of 133 mg (0.62 mmol, 62%) was obtained.

### EXAMPLE 13

### Comparison of the activity of sulfonated and unsulfonated 2-hydroxy-2′-dicyclohexylphosphinobiphenyl

207 mg of 1-bromonaphthalene (1 mmol), 118 mg of 4 aminobenzonitrile (1 mmol), 192 mg (2 mmol) of sodium tert-butoxide, 2.2 mg of palladium(II) acetate (1 mol %) and 4.5 mg of the HBPNS ligand (1 mol %) were heated to 120° C. in 6 ml of degassed diglyme for 29 h. In parallel, an identical experiment was performed, in which the unsulfonated ligand 2′-hydroxy-2-dicyclohexylphosphinobiphenyl (3.7 mg, 1 mol %) was used. At regular intervals (see table), samples were taken from the two reactions and analyzed by GC:

| | Conversion (sulfonated | Conversion (unsulfonated |
|---|---|---|
| Time/min | ligand) | ligand) |
| 180 | 18% | 7% |
| 240 | 29% | 12% |
| 300 | 48% | 18% |
| 540 | 71% | 34% |
| 620 | 89% | 48% |
| 1380 | 93% | 79% |

After 29 h, the reaction was stopped. The isolated yields were significantly higher in the case of the sulfonated ligand, but the reaction is in particular much more rapid in the case of the sulfonated ligand (see FIG. 1).

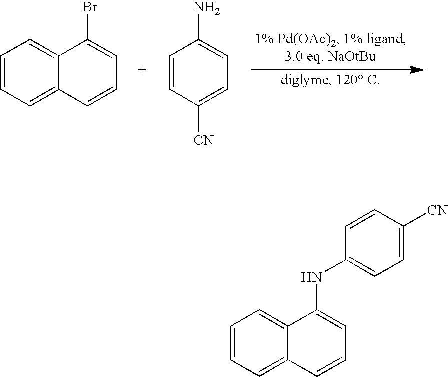

The lower reaction rate in the case of the “classical” ligand is seen clearly. For instance, almost 90% conversion has already been achieved with the sulfonated ligand after 635 min (corresponds to 5 h 35 min), but not even 50% with the unsulfonated ligand. Only after a very long reaction time do the conversions slowly converge.

1. A process for preparing aryl- or heteroarylamines, aryl or heteroaryl ethers or aryl or heteroaryl thioethers (III) comprising cross-coupling primary or secondary amines, alcohols or thioalcohols (II) with substituted aryl or heteroaryl compounds (I), in the presence of a Brønsted base and of a catalyst or precatalyst comprising
a.) a transition metal, a complex, a salt or a compound of this transition metal from the group of V, Mn, Fe, Co, Ni, Rh, Pd, Ir, Pt, and
b.) at least one sulfonated phosphine ligand
in a solvent or solvent mixture, according to scheme 1 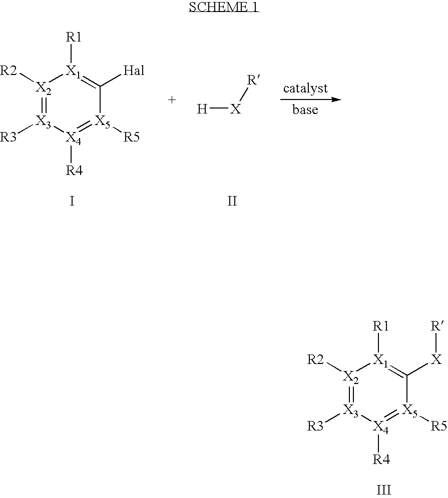
where Hal is fluorine, chlorine, bromine, iodine, alkoxy, trifluoromethanesulfonate, nonafluorotrimethylmethanesulfonate, methanesulfonate, 4-toluenesulfonate, benzenesulfonate, 2-naphthalenesulfonate, 3-nitrobenzenesulfonate, 4-nitrobenzenesulfonate, 4-chlorobenzenesulfonate, 2,4,6-triisopropylbenzenesulfonate or any other sulfonate,
X is O, S or NR″,
X₁₋₅ are each independently carbon, or XᵢRᵢ is nitrogen, or in each case two adjacent XᵢRᵢ bonded via a formal double bond together are O, S, NH or NRᵢ, the R₁₋₅ radicals are substituents from the group of hydrogen, methyl, primary, secondary or tertiary, cyclic or acyclic alkyl radicals having from 2 to 20 carbon atoms, in which one or more hydrogen atoms are optionally replaced by fluorine or chlorine or bromine, substituted cyclic or acyclic alkyl groups, hydroxyl, alkoxy, amino, alkylamino, dialkylamino, arylamino, diarylamino, alkylarylamino, pentafluorosulfanyl, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻, alkyl- or aryloxycarbonyl, hydroxyalkyl, alkoxyalkyl, fluorine or chlorine, nitro, cyano, aryl- or alkylsulfone, aryl- or alkylsulfonyl or in each case two adjacent R₁₋₅ radicals together are an aromatic, heteroaromatic or aliphatic fused-on ring,
when X═O or S,R′ may be identical or different radicals from the group of hydrogen, methyl, linear, branched C₁-C₂₀ alkyl or cyclic alkyl, substituted or unsubstituted aryl or heteroaryl,
or X is NR″, where R′ and R″ are each independently identical or different radicals from the group of hydrogen, methyl, linear, branched C₁-C₂₀ alkyl or cyclic alkyl, substituted or unsubstituted aryl or heteroaryl or together form a ring,
and wherein a sulfonated phosphine ligand of the structure 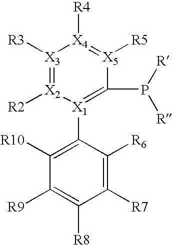
is used, where
X₁ is carbon or nitrogen, X₂₋₅ are each independently carbon, or XᵢRᵢ is nitrogen, or in each case two adjacent XᵢRᵢ bonded via a formal double bond, where i=2, 3, 4, 5, together are O, S, NH or NRᵢ,
the R₂₋₁₀ radicals, where at least one radical is a sulfonic acid or sulfonate group, are each substituents from the group of hydrogen, methyl, primary, secondary or tertiary, cyclic or acyclic alkyl radicals having from 2 to 20 carbon atoms, in which one or more hydrogen atoms are optionally replaced by fluorine or chlorine or bromine, substituted cyclic or acyclic alkyl groups, hydroxyl, alkoxy, amino, alkylamino, dialkylamino, arylamino, diarylamino, alkylarylamino, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻, alkyl- or aryloxycarbonyl, hydroxyalkyl, alkoxyalkyl, nitro, cyano, aryl- or alkylsulfone, aryl- or alkylsulfonyl, or two adjacent R₂₋₅ radicals together are an aromatic, heteroaromatic or aliphatic fused-on ring,
R′ and R″ are each independently identical or different radicals from the group of hydrogen, methyl, linear, branched or cyclic alkyl, phenyl or together form a ring and are a bridging structural element from the group of alkylene, branched alkylene, cyclic alkylene, or are each independently one or two polycyclic radicals.

2. The process as claimed in claim 1, wherein the sulfonated phosphine ligands contain at least one sulfonic acid group or a metal sulfonate.

3. The process as claimed in claim 1, wherein the Brønsted base is a hydroxide, alkoxide or amide of the alkali metals or alkaline earth metals or an alkali metal carbonate or phosphate or mixtures of these compounds.

4. The process as claimed in claim 1, wherein the cross-coupling comprises from 1.0 to 3 equivalents of base based on the aryl or heteroaryl halide or aryl or heteroaryl sulfonate.

5. The process as claimed in claim 1, wherein the solvents are hydrocarbons, halogenated hydrocarbons, open-chain and cyclic ethers and diethers, oligo- and polyethers, tertiary amines, DMSO, NMP, DMF, DMAc, and substituted mono- or polyalcohols and optionally substituted aromatics, or a mixture of a plurality of these solvents.

6. The process as claimed in claim 1, wherein the process is performed at a temperature in the range from 0 to 240° C.

7. The process as claimed in claim 1, wherein the cross-coupling comprises catalyst in a ratio relative to the reactant (I) in amounts of from 0.001 mol % to 100 mol %.

8. The process as claimed in claim 1, wherein the cross-coupling comprises a complex of a sulfonated secondary phosphine in conjunction with a palladacycle as a catalyst of the structure 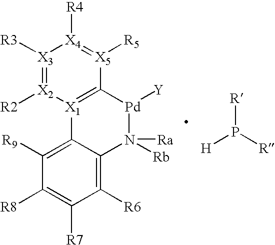
where the symbols X₁₋₅, R₂₋₉, R′ and R″ are each as defined in claim 1 and Y is a radical from the group of halide, pseudohalide, alkylcarboxylate, trifluoroacetate, nitrate, nitrite, and Rₐ and R_{b} are each independently identical or different substituents from the group of hydrogen, methyl, primary, secondary or tertiary, optionally substituted alkyl or aryl or together form a ring and stem from the group of optionally substituted alkylene, oxaalkylene, thiaalkylene, azaalkylene.

9. The process as claimed in claim 1, wherein the cross-coupling comprises a complex of a sulfonated tertiary phosphine of the structure 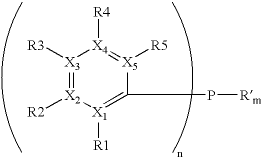
where the symbols X₁₋₅, R₁₋₅ and R′ are each as defined in claim 1, where n may be 1, 2 or 3 and m=3−n, and the n aryl or heteroaryl radicals and the m radicals may each independently be the same or different, and further optionally comprising mixtures of different ligands of this class.

10. A sulfonated ligand of the structure 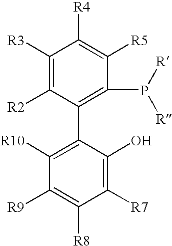
in which at least one R₁ radical represents a sulfonic acid or sulfonate group and the R₂₋₅ and R₇₋₁₀ radicals are each substituents for the group of hydrogen, methyl, primary, secondary or tertiary, cyclic or acyclic alkyl radicals having from 2 to 20 carbon atoms, in which one or more hydrogen atoms are optionally replaced by fluorine or chlorine or bromine, substituted cyclic or acyclic alkyl groups, hydroxyl, alkoxy, amino, alkylamino, dialkylamino, arylamino, diarylamino, alkylarylamino, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂⁻, alkyl- or aryloxycarbonyl, hydroxyalkyl, alkoxyalkyl, nitro, cyano, aryl- or alkylsulfone, aryl- or alkylsulfonyl, or two adjacent R₂₋₅ radicals together are an aromatic, heteroaromatic or aliphatic fused-on ring,
R′ and R″ are each independently identical or different radicals from the group of hydrogen, methyl, linear, branched or cyclic alkyl, phenyl or together form a ring and are a bridging structural element from the group of alkylene, branched alkylene, cyclic alkylene, or are each independently one or two polycyclic radicals.

11. A sulfonated ligand of the structure 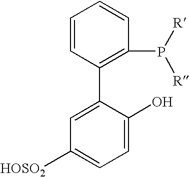
in which R′ and R″ are each independently identical or different radicals from the group of hydrogen, methyl, linear, branched or cyclic alkyl, phenyl or together form a ring and are a bridging structural element from the group of alkylene, branched alkylene, cyclic alkylene or are each independently one or two polycyclic radicals.

12. A complex, mixture, salt or formulation comprising at least one ligand as claimed in claim 10 and at least one metal, metal complex, metal salt or metal compound from the group of V, Mn, Fe, Co, Ni, Rh, Pd, Ir, Pt.

13. Catalysts in organochemical catalysis comprising ligands as claimed in claim 10.

14. A process for preparing sulfonated 2-hydroxy-2′phosphinobiphenyls comprising subjecting 2-hydroxy-2′-phosphinobiphenyls to electrophilic sulfonation.

15. A process for preparing sulfonated 2-hydroxy-2′-phosphinobiphenyls from 2-hydroxy-2′-phosphinobiphenyls comprising performing a metallation reaction and subsequent quenching with a sulfonation reagent.

16. The process as claimed in claim 1, wherein R′ and R″ are each independently identical or different radicals from the group of hydrogen, methyl, linear, branched or cyclic alkyl, phenyl or together form a ring and are a bridging structural element from the group of alkylene, branched alkylene, cyclic alkylene, or are each independently norbornyl or adamantyl.

17. A sulfonated ligand as claimed in claim 10, wherein R′ and R″ are each independently identical or different radicals from the group of hydrogen, methyl, linear, branched or cyclic alkyl, phenyl or together form a ring and are a bridging structural element from the group of alkylene, branched alkylene, cyclic alkylene, or are each independently norbornyl or adamantyl.

18. A sulfonated ligand as claimed in claim 11, wherein R′ and R″ are each independently identical or different radicals from the group of hydrogen, methyl, linear, branched or cyclic alkyl, phenyl, or together form a ring and are a bridging structural element from the group of alkylene, branched alkylene, cyclic alkylene or are each independently norbornyl or adamantyl.

19. A complex, mixture, salt or formulation comprising at least one ligand as claimed in claim 11 and at least one metal, metal complex, metal salt or metal compound from the group of V, Mn, Fe, Co, Ni, Rh, Pd, Ir, Pt.

20. Catalysts in organochemical catalysis comprising ligands as claimed in claim 11.
